(19) United States
(12) Patent Application Publication
(10) Pub. No.: US 2021/0095246 A1
(43) Pub. Date: Apr. 1, 2021

(54) STORAGE AND/OR TRANSPORT FOR MULTICELLULAR AGGREGATES

(71) Applicant: ATELERIX LIMITED, Cambridge (GB)

(72) Inventors: Stephen SWIOKLO, Tyne and Wear (GB); Che John CONNON, Tyne and Wear (GB)

(73) Assignee: ATELERIX LIMITED, Cambridge (GB)

(21) Appl. No.: 15/733,394

(22) PCT Filed: Jan. 21, 2019
(86) PCT No.: PCT/GB2019/050158

§ 371 Date: Jul. 20, 2020

(30) Foreign Application Priority Data
Jan. 22, 2018 (GB) 1801014.0

(51) Int. Cl. C12N 5/00; C12N 5/0775; C12N 5/0793; C12N 5/071

(52) U.S. Cl. CPC C12N 5/0012; C12N 5/0667; C12N 2533/74; C12N 5/0686; C12N 5/0619

(57) ABSTRACT

The present invention provides a novel means for storing and/or transporting multicellular aggregates. The multicellular aggregates comprise a plurality of adjoining cells, wherein the aggregate is entrapped or encapsulated in a reversibly cross-linked hydrogel and the entrapped or encapsulated aggregate is packaged in a sealed receptacle. Methods for preparing such aggregates for storage and/or transportation from a first location to a second location are also provided, together with related methods for transporting or storing said aggregates and methods for fulfilling an order or request for said aggregates.

The present invention provides a novel means for storing and/or transporting multicellular aggregates. The multicellular aggregates comprise a plurality of adjoining cells, wherein the aggregate is entrapped or encapsulated in a reversibly cross-linked hydrogel and the entrapped or encapsulated aggregate is packaged in a sealed receptacle. Methods for preparing such aggregates for storage and/or transportation from a first location to a second location are also provided, together with related methods for transporting said aggregates and methods for fulfilling an order or request for said aggregates.

### BACKGROUND

Cells may be used in several contexts, including scientific research, foodstuff, drug development, regenerative medicine and 3D printing. Appropriate cells may be in the form of a group of adjoining cells (generally referred to herein as multicellular aggregates), which include tissues (e.g. micro-tissues), cell layers, organoids and spheroids.

Multicellular aggregates may be generated and/or prepared for use in a location that is often geographically separated from their point of use. However, shipping of such cellular materials within the UK or globally can take hours or days and is vulnerable to delays, and the material needs to be delivered to the point of use in a condition that is fit for purpose. Effective transportation and recovery of multicellular aggregates such as tissues has proven difficult, with many methods resulting in changes in e.g. cellular morphology, cell integrity and/or loss of cell viability over time. Storage and/or transport of multicellular aggregates therefore represents a significant barrier in respect of e.g. laboratory supply (distribution for research) and therapeutics (commercial sale/trials).

Conventional methods for the storage and shipment of cellular materials are either cold-chain shipping in appropriate media (e.g. at 2-8° C.) or freezing the sample prior to, and during, shipping. For example, transportation of cryopreserved tissue is commonly used. However, such methods generally require that a number of processing steps are carried out prior to shipping, and these processes may adversely affect the shipped material or significantly increase cost. For example, cryopreservation often leads to loss in cell or tissue viability, reduced structural integrity and added expense through the necessity to maintain low temperatures during transport. Cold-chain shipping also has a number of drawbacks. These include the need for reduced transport duration (thus increasing the complication of shipping logistics and scheduling), and adverse effects on cell or tissue viability, morphology, structural integrity and quality. These drawbacks are a particularly significant problem when the cellular material is for human use (e.g. for cosmetic or clinical use, or for human use as a foodstuff).

There is a need for a simple yet effective method for storing and/or transporting cellular material including multicellular aggregates.

### BRIEF SUMMARY OF THE DISCLOSURE

The inventors have developed a novel means for storing and/or transporting multicellular aggregates that comprise a plurality of adjoining cells.

The inventors have surprisingly shown that the entrapment or encapsulation of a multicellular aggregate in a reversibly cross-linked hydrogel protects the cellular material in the aggregate from the mechanical and environmental stresses of storage and/or transportation. Surprisingly, the entrapped or encapsulated cellular material does not require the optimum conditions normally required to maintain cell morphology, structural integrity and/or cell viability (e.g. a certain temperature, oxygen and carbon dioxide level, and supporting nutrients) during storage and/or transportation. Accordingly, the entrapped or encapsulated cellular material can be packaged in a sealed receptacle for effective storage or delivery to its point of use, whilst maintaining the material in a condition that is fit for purpose. Furthermore, storage and/or transportation of the packaged material can effectively be undertaken at a much broader range of conditions (e.g. a broader range of temperatures, including ambient temperature) without significantly impacting cellular viability, structural integrity and/or morphology.

Hydrogels have previously been shown to be an effective packaging material for use in the storage and/or transportation of individualised cells, wherein the cells are separated or dispersed within the hydrogel (see for example WO 2012/127224, filed by the inventors). The inventors have now surprisingly identified that each cell does not need to be individually in direct contact with the hydrogel for the hydrogel to provide the necessary protection from mechanical and environmental stresses, including stress from lack of soluble factors such as gases and metabolites, during storage and/or transportation. The inventors have advantageously shown herein that hydrogels can also be used to support the viability (and retain the cellular morphology and structural integrity) of multicellular aggregates comprising a plurality of adjoining cells during storage and/or transport. Examples of the types of aggregates that have successfully been tested by the inventors include cellular spheroids, organoids, micro-tissues and cell layers (e.g. multicellular aggregates having at least one layer, wherein the basal layer/side of the aggregate is adherent to a tissue culture plate on one side, and the apical layer/side of the aggregate is coated with the hydrogel on the other side). In this context, the aggregate may comprise one cell layer (i.e. a monolayer) or may comprise a plurality of layers (e.g. a bilayer etc).

Advantageously, the hydrogel can be used effectively to store and/or transport a broad range of multicellular aggregates.

The methods of the invention may be particularly useful for storing multicellular material (such as isolated or manufactured tissues) immediately, before any cellular deterioration has occurred and this provides flexibility to the user, as the multicellular material (e.g. isolated/manufactured tissue) can be safely stored until the appropriate staff are available, a GMP laboratory is accessible or until samples can be processed in bulk, without impacting endpoint performance.

The invention has been exemplified using alginate hydrogels. However, the invention applies equally to other reversibly cross-linked hydrogels with the equivalent mechanical properties. Alternative hydrogels that may be equally used within the context of the invention are described in more detail below.

Furthermore, the invention has been exemplified using certain cell types e.g. multicellular aggregates comprising stromal cells, epithelial cells or neuronal cells. In addition, data is presented describing the use of the invention on simple multicellular spheroids and simple 3D tissue constructs. However, the invention is not limited to these particular cell types and is equally applicable to other multicellular aggregates, as described in more detail below.

In one aspect, there is provided a method of transporting an in vitro multicellular aggregate comprising a plurality of adjoining cells from a first location to a second location, the method comprising the steps of:

(a) preparing the multicellular aggregate for transportation by;
i) contacting the multicellular aggregate with an alginate hydrogel-forming polymer;
ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
   (b) transporting the packaged multicellular aggregate of step (a) from the first location to the second location at a temperature from 10 to 30° C., wherein the distance between the first and second location is at least 1 mile.

Optionally, the method may further comprise:

(c) releasing the multicellular aggregate from the alginate hydrogel at the second location.

In another aspect, there is provided a method for fulfilling an order or request for an in vitro multicellular aggregate comprising a plurality of adjoining cells, the method comprising: receiving an order or request for a multicellular aggregate; and

a) preparing the multicellular aggregate for transportation by;
i) contacting the multicellular aggregate with an alginate hydrogel-forming polymer;
ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
   b) dispatching the packaged multicellular aggregate of step (a) for transportation; or transporting the multicellular aggregate of step (a) to the location specified in the order or request.

Optionally, the multicellular aggregate is transported from the first location to the second location at a temperature from 10 to 30° C. and the distance between the first and second location is at least 1 mile.

In a further aspect, there is provided a method of storing an in vitro multicellular aggregate comprising a plurality of adjoining cells for at least 24 hours, the method comprising the steps of:

(a) preparing the multicellular aggregate for storage by;
i) contacting the multicellular aggregate with an alginate hydrogel-forming polymer;
ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
   (b) storing the packaged multicellular aggregate of step (a) for at least 24 hours at a temperature from 10 to 30° C.

Optionally, the method may further comprise: (c) releasing the multicellular aggregate from the alginate hydrogel after storage.

Optionally, step (a) comprises placing the multicellular aggregate in the receptacle for transportation, dispatch or storage prior to contacting the multicellular aggregate with the alginate hydrogel-forming polymer.

Alternatively, step (a) comprises placing the multicellular aggregate in the receptacle for transportation, dispatch or storage after contacting the multicellular aggregate with the alginate hydrogel-forming polymer.

Optionally, the receptacle is a cell culture vessel.

Optionally, the cell culture vessel is selected from a cell culture tube, a cell culture flask, a cell culture dish or a cell culture plate comprising a plurality of wells.

Optionally, the cell culture plate comprising a plurality of wells is selected from a 4-, 6-, 8-, 12-, 24-, 48-, 96-, 384-, 1536-well cell culture plate.

Optionally, the hydrogel-forming polymer comprises calcium-alginate, strontium alginate, barium-alginate, magnesium-alginate or sodium-alginate.

Optionally, the alginate is in an amount from 0.5% (w/v) to 5.0% (w/v) calcium alginate.

Optionally, the multicellular aggregate comprises a tissue, a cell layer, a spheroid, an organoid or any combination thereof.

Optionally, the multicellular aggregate comprises heterogenous cell types.

Alternatively, the multicellular aggregate comprises homogenous cell types.

Optionally, the multicellular aggregate comprises human cells.

Optionally, the multicellular aggregate comprises human adipose-derived stromal cells (hASCs), human induced-pluripotent stem cells (iPSC)-derived cortical neurons, human primary kidney proximal tubule epithelial cells (hPTCs), or human corneal stromal fibroblasts (hCSF).

Optionally, polymerisation is induced by a chemical agent.

Optionally, the chemical polymerisation agent is calcium chloride.

In another aspect, there is provided an in vitro tissue comprising a plurality of adjoining cells, wherein the tissue is entrapped or encapsulated in a reversibly cross-linked alginate hydrogel and the entrapped or encapsulated tissue is packaged in a sealed water tight or air tight receptacle.

Optionally, the hydrogel comprises cross-linked calcium-alginate, strontium-alginate, barium-alginate, magnesium-alginate or sodium-alginate.

Optionally, the cross-linked alginate is from 0.5% (w/v) to 5.0% (w/v) calcium alginate.

Optionally, the plurality of adjoining cells form a cell layer, a spheroid, an organoid or any combination thereof.

Optionally, the receptacle is a sealed storage vial or transport tube.

Optionally, the sealed storage vial is a microcentrifuge tube, centrifuge tube, cryogenic vial, transport tube, or universal container.

Optionally, the receptacle is a cell culture vessel.

Optionally, the cell culture vessel is selected from a cell culture tube, a cell culture flask, a cell culture dish or a cell culture plate comprising a plurality of wells.

Optionally, the cell culture plate comprising a plurality of wells is selected from a 4-, 6-, 8-, 12-, 24-, 48-, 96-, 384-, 1536-well cell culture plate.

Optionally, the multicellular aggregate comprises heterogenous cell types.

Alternatively, the multicellular aggregate comprises homogenous cell types.

Optionally, the multicellular aggregate comprises human cells.

Optionally, the multicellular aggregate comprises human adipose-derived stromal cells (hASCs), human induced-pluripotent stem cells (iPSC)-derived cortical neurons, human primary kidney proximal tubule epithelial cells (hPTCs), or human corneal stromal fibroblasts (hCSF).

In a further aspect, there is provided a method of preparing an in vitro tissue comprising a plurality of adjoining cells for storage or transportation from a first location to a second location, the method comprising the steps of:
i) contacting the tissue with an alginate hydrogel-forming polymer;
ii) polymerising the polymer to form a reversibly cross-linked tissue-containing alginate hydrogel wherein the tissue is entrapped or encapsulated in the alginate hydrogel; and
iii) packaging and sealing the multicellular aggregate-containing alginate hydrogel in a water tight or air tight receptacle.

Optionally, the method comprises placing the tissue in the receptacle for storage or transportation prior to contacting the tissue with the alginate hydrogel-forming polymer.

Optionally, the method comprises placing the tissue in the receptacle for storage or transportation after contacting the tissue with the alginate hydrogel-forming polymer.

Optionally, the method further comprises iii) dispatching the sealed receptacle for transportation from the first location to the second location, wherein the multicellular aggregate is transported from the first location to the second location at a temperature from 10 to 30° C. and the distance between the first and second location is at least 1 mile.

In another aspect, there is provided a multicellular aggregate comprising a plurality of adjoining cells, wherein the aggregate is entrapped or encapsulated in a reversibly cross-linked hydrogel and the entrapped or encapsulated aggregate is packaged in a sealed receptacle.

Optionally, the hydrogel comprises cross-linked alginate, wherein the hydrogel optionally comprises cross-linked calcium-alginate, strontium-alginate, barium-alginate, magnesium-alginate or sodium-alginate.

Optionally, the cross-linked alginate is from about 0.5% (w/v) to 5.0% (w/v) calcium alginate.

Optionally, the plurality of adjoining cells form a tissue, a cell layer, a spheroid, an organoid or any combination thereof.

Optionally, the receptacle is a cell culture vessel.

Optionally, the cell culture vessel is selected from a cell culture tube, a cell culture flask, a cell culture dish or a cell culture plate comprising a plurality of wells.

Optionally, the cell culture plate comprising a plurality of wells is selected from a 4-, 6-, 8-, 12-, 24-, 48-, 96-, 384-, 1536-well cell culture plate.

In another aspect, there is provided a method of preparing a multicellular aggregate comprising a plurality of adjoining cells for storage or transportation from a first location to a second location, the method comprising the steps of:

i) contacting the multicellular aggregate with a hydrogel-forming polymer;
ii) polymerising the polymer to form a reversibly cross-linked aggregate-containing hydrogel wherein the aggregate is entrapped or encapsulated in the hydrogel;
wherein the aggregate-containing hydrogel is packaged in a receptacle for storage or transportation from the first location to the second location and wherein the method comprises sealing the aggregate-containing hydrogel into the receptacle.

Optionally, the method comprises placing the multicellular aggregate in the receptacle for storage or transportation prior to contacting the multicellular aggregate with the hydrogel-forming polymer.

Alternatively, the method comprises placing the multicellular aggregate in the receptacle for storage or transportation after contacting the multicellular aggregate with the hydrogel-forming polymer.

Optionally, the method further comprises dispatching the sealed receptacle for transportation from the first location to the second location.

In another aspect, there is provided a method of transporting a multicellular aggregate comprising a plurality of adjoining cells from a first location to a second location, the method comprising the steps of:

(a) preparing the multicellular aggregate for transportation according to the methods described herein;
(b) transporting the multicellular aggregate of step (a) from the first location to the second location; and optionally
(c) releasing the multicellular aggregate from the hydrogel at the second location.

In another aspect, there is provided a method for fulfilling an order or request for a multicellular aggregate, the method comprising the steps of:

a) receiving an order or request for a multicellular aggregate;
b) preparing the multicellular aggregate for transportation according to the methods described herein; and
c) dispatching the multicellular aggregate of step (b) for transportation; or transporting the multicellular aggregate of step (b) to the location specified in the order or request.

Optionally, the receptacle is a cell culture vessel.

Optionally, the cell culture vessel is selected from a cell culture tube, a cell culture flask, a cell culture dish or a cell culture plate comprising a plurality of wells.

Optionally, the cell culture plate comprising a plurality of wells is selected from a 4-, 6-, 8-, 12-, 24-, 48-, 96-, 384-, 1536-well cell culture plate.

Optionally, the hydrogel comprises alginate.

Optionally, the hydrogel-forming polymer comprises calcium-alginate, strontium-alginate, barium-alginate, magnesium-alginate or sodium-alginate.

Optionally, the alginate is in an amount from about 0.5% (w/v) to 5.0% (w/v) calcium alginate.

Optionally, the multicellular aggregate comprises a tissue, a cell layer, a spheroid, an organoid or any combination thereof.

Optionally, the multicellular aggregate comprises heterogenous or homogenous cell types.

Optionally, the multicellular aggregate comprises human cells.

Optionally, the multicellular aggregate comprises human adipose-derived stromal cells (hASCs), human induced-pluripotent stem cells (iPSC)-derived cortical neurons, human primary kidney proximal tubule epithelial cells (hPTCs), or human corneal stromal fibroblasts (hCSF).

Optionally, polymerisation is induced by a chemical agent.

Optionally, the chemical polymerisation agent is calcium chloride.

Optionally, the multicellular aggregate is transported from the first location to the second location at ambient temperature.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### BRIEF DESCRIPTION OF THE DRAWINGS

Embodiments of the invention are further described hereinafter with reference to the accompanying drawings, in which:

FIG. 1 shows cell recovery, viability and morphology of human adipose-derived mesenchymal stromal cells (hASCs) following storage of cell monolayers in 96-well plates, with or without alginate hydrogel protection.

FIG. 2 shows cell recovery, viability and morphology of mature cortical neurons following storage and shipment in 96-well plate, with or without alginate hydrogel protection.

FIG. 3 shows cell recovery, viability and morphology of primary human kidney proximal tubule epithelial cells (hPTCs) following storage in 96-well plates, with or without alginate hydrogel protection.

FIG. 4 shows viability of hASC-derived spheroids following storage in tightly-sealed tubes, with or without alginate hydrogel protection. Within graph, right hand bar at each time point corresponds to ‘+ Hydrogel’. No cellular outgrowth is seen when spheroids are plated on to tissue culture plastic following storage in ‘− Hydrogel’.

FIG. 5 shows viability of hASC-derived spheroids following storage in 96-well plates, with or without alginate hydrogel protection. A) a single well from a 96 well plate.

FIG. 6 shows viability and integrity of human corneal stromal fibroblast (hCSF) constructs in tightly-sealed tubes, with or without alginate hydrogel protection. It is noted that no viable cells are seen remaining in the − Hydrogel storage condition.

FIG. 7 shows the storage of dermal keratinocyte epithelial cells preserved in 96-well culture plates. The viability and morphology of human dermal keratinocyte epithelial cells were preserved in 96-well culture plates.

FIG. 8 shows the storage and shipment of dermal fibroblast cells preserved in 96-well culture plates. The viability and morphology of human dermal fibroblast cells were preserved in 96-well culture plates.

FIG. 9 shows the storage and shipment of HEK-293 cells preserved in 96-well culture plates, 384-well culture plates, and 3D microscaffolds in 96-well plates. The pharmacological responsiveness of HEK-293 and transiently transfected HEK-293 cells were preserved.

FIG. 10 shows the storage of human abdominal skin biopsies in 96-well plates. Freshly collected abdominal skin biopsies in 96-well plates were preserved.

FIG. 11 shows the storage of iPSC-derived hemangioblasts (macrophage progenitor factories). iPSC-derived hemangioblasts suspended in calcium alginate hydrogel beads were preserved.

FIG. 12 shows the storage of human skin 3D constructs. The human skin 3D constructs were preserved with alginate hydrogel protection.

FIG. 13 shows the storage of Colorectal Cancer Organoids preserved in 96-well culture plates. The viability and morphology of colorectal cancer organoids were preserved following storage in 96-well plates with alginate hydrogel protection.

### DETAILED DESCRIPTION

Several different aspects of the invention are described below. They are discussed separately for ease of understanding. However, each of the definitions and examples provided applies to all aspects equally, where the context allows.

### Multicellular Aggregates

A multicellular aggregate is provided comprising a plurality of adjoining or interconnected cells, wherein the aggregate is entrapped or encapsulated in a reversibly cross-linked hydrogel and the entrapped or encapsulated aggregate is packaged in a sealed receptacle.

It has been shown previously that completely encapsulating individual cells in an alginate hydrogel can preserve their functionality at hypothermic temperatures. In a multicellular aggregate however, each cell is not completely encapsulated in the hydrogel since at least one surface (or part of a surface) of each cell is in contact with another cell (or a matrix or an artificial construct). In a three-dimensional multicellular aggregate some cells in the interior may not be encapsulated by the hydrogel at all while those towards the exterior will be somewhat encapsulated. The inventors have now surprisingly shown that encapsulation or entrapment of multicellular aggregates in a hydrogel as described herein, wherein each cell of the aggregate is not completely and directly surrounded by the hydrogel itself can be used to effectively store and/or transport multicellular aggregates whilst retaining cell morphology, integrity and/or viability.

The terms “multicellular aggregate” and “aggregate” are used interchangeably herein, unless the context specifies otherwise. An aggregate refers to e.g. a ball, cluster, layer etc of cells.

As used herein, “multicellular aggregate” refers to a plurality of adjoining or interconnected cells. A multicellular aggregate may be formed from e.g. at least 10 adjoining cells (wherein each cell is in direct contact (in other words touching) with at least one other cell within the aggregate). For example, the aggregate may comprise at least 10, at least 10², at least 10³, at least 10⁴, at least 10⁵, at least 10⁶, at least 10⁷, at least 10⁸, or at least 10⁹ etc adjoining cells. In a preferred example, the adjoining cells are interconnected.

Optionally, the multicellular aggregate is an in vitro multicellular aggregate (in other words the multicellular aggregate is isolated and outside of its biological context).

The cells of the multicellular aggregate typically have a structurally intact cell membrane. Several methods for determining the structural integrity of a cell membrane are known, including propidium iodide staining (see examples below).

In a preferred example, the cells in the multicellular aggregate are viable or living cells, or at least substantially all of the cells in the multicellular aggregate are preferably live (or viable). Methods for determining whether or not cells are living are well known in the art.

As used herein, “adjoining” refers to cells that are connected to each other in a manner that forms an aggregate of cells. The adjoining cells retain the aggregate form when placed in a solution such as a hydrogel forming polymer solution. Adjoining cells may be in direct contact e.g. wherein they adhere to or touch each other in a manner that forms an aggregate of cells. Alternatively, adjoining cells may be connected indirectly in a manner that forms an aggregate of cells, such as by virtue of the presence of a matrix, substrate or scaffold (e.g. an extracellular matrix), wherein the matrix, substrate or scaffold connects the adjoining cells into the aggregate.

As described above, a matrix, substrate or scaffold may connect adjoining cells, to form an aggregate. The terms “matrix”, “substrate” and “scaffold” used interchangeably herein, and are generically referred to as a “structure” within the aggregate. It has been found that the mechanical strength of the hydrogel may be enhanced by the encapsulation of such structure within the gel. The structure may also facilitate or maintain aggregate formation. The structure may be naturally derived or synthetic.

In one example, the structure may be a synthetic or natural polymer. Preferably, the structure is biodegradable. The structure may, for example be a polymer comprising polylactic acid (e.g. poly(lactic acid-co-caprolactone) (PLACL)), collagen or nylon.

In another example, the cells are adjoined via an extracellular matrix (ECM) in a manner that forms a multicellular aggregate. A further example of a suitable structure is an Alvatex® polystyrene scaffold for 3D cell culture. Other structures may comprise collagen, gelatin, alginate, cellulose, glass, or matrigel, etc.

The structure may also be a nylon mesh. Such a composite material has the advantage of being more robust than an alginate gel and less likely to break up during storage or transit of the gel. A further benefit is that the nylon mesh may be sutured, thereby allowing the gel to be held by stitches. The nylon mesh may be within the gel, partially within and partially outside the gel or outside (i.e. on a surface of) the gel. The nylon mesh preferably has a mesh size of 0.01-100 μm. Preferably, it is made of a suitable non-toxic material, which may be soluble or insoluble. In a preferred example, the hydrogel is in the form of a disc comprising a nylon mesh. Preferably, the nylon mesh is embedded within the disc.

Alternatively, the aggregates may be structure-free. Appropriate methods for cell culture with or without a structure are well known in the art.

In a preferred example, the adjoining cells are interconnected. As used herein, “interconnected” refers to cells that are in direct contact with each other and are physically connected e.g. by intercellular connections (e.g. by one or more cell junction(s) (also known as intercellular bridge(s))). Cell junctions are made up of multiprotein complexes that provide contact between neighboring cells or between a cell and the extracellular matrix. Cell junctions are especially abundant in epithelial tissues. Cell junctions enable communication between neighbouring cells.

The multicellular aggregate may be any group of adjoining cells, for example, it may be in the form of a tissue or an organ (e.g. an animal or plant tissue or organ, or a synthetic/artificial tissue or organ i.e. tissue engineered tissue or organ).

Examples of suitable animal tissues or organs include skin, cornea, muscle, liver, and heart tissues or organs. Such tissues or organs may be obtained directly from a living animal. Methods for isolating appropriate multicellular aggregates from animals are well known in the art.

Examples of suitable plant tissues or organs (that are obtained from a living plant) include cells or tissues derived from the endoderm, mesoderm and ectoderm germ layers, mesophyll tissue, xylem tissue and phloem tissue, leaf, stem, root, and reproductive organs.

Methods for isolating appropriate multicellular aggregates from plants are well known in the art.

Examples of suitable synthetic tissue or organs include any cellular tissues or organs that have been generated or propagated in vitro or ex vivo. Non-limiting examples include cellular spheres, spheroids, organoids or micro-tissues. These types of aggregates are typically generated using cell culture methods in three-dimensions. Such methods are well known in the art. Examples of appropriate methods are provided in the examples section below.

Multicellular aggregates described herein may also comprise a plurality of adjoining (e.g. interconnected) cells, wherein the cells are in the form of a sheet of cells (i.e. one or more layer(s) of cells, such as a monolayer), for example, a sheet of cells that has been cultured in vitro or ex vivo. In other words, the aggregate may be planar. A non-limiting example would be a multicellular aggregate comprising a sheet of corneal cells (e.g. a monolayer of corneal cells). Examples of appropriate methods are provided in the examples section below.

In one example, the multicellular aggregate may be attached to a surface (e.g. to a surface of a receptacle such as a tissue culture well or a tissue culture flask). For example, the multicellular aggregate may comprise adherent cells and the adherent cells may adhere to a surface of a receptacle. Appropriate receptacles (such as sealable receptacles) are described in detail elsewhere herein. In one example, the multicellular aggregate comprises cells that form an adherent layer (e.g. a monolayer, bilayer or multilayer aggregate) on such a surface.

In one example, the multicellular aggregate may be attached to a surface of a receptacle (e.g. culture vessel) in which they were seeded and/or grown in vitro.

In one particular example, the multicellular aggregate comprises a plurality of adjoining (e.g. interconnected) cells, wherein the cells form a tissue, a cell layer, a spheroid, an organoid or any combination thereof.

In some examples, the cells in the multicellular aggregate are all of the same type. For example, they may all be brain cells, muscle cells or heart cells. In other examples, the cells in the multicellular aggregate are all from the same lineage, e.g. all haematopoietic precursor cells. In some examples, the cells are stem cells, for example, neural stem cells or embryonic stem cells.

Accordingly, in one example, a multicellular aggregate comprises homogeneous or heterogeneous cell types.

In an example, the cells are adipocytes, astrocytes, blood cells, blood-derived cells, bone marrow cells, bone osteosarcoma cells, brain astrocytoma cells, breast cancer cells, cardiac myocytes, cerebellar granule cells, chondrocytes, corneal cells, dermal papilla cells, embryonal carcinoma cells, embryonic stem cells, embryo kidney cells, endothelial cells, epithelial cells, erythroleukaemic lymphoblasts, fibroblasts, foetal cells, germinal matrix cells, hepatocytes, intestinal cells, keratinocytes, keratocytes, kidney cells, liver cells, lung cells, lymphoblasts, melanocytes, mesangial cells, meningeal cells, mesenchymal stem cells, microglial cells, neural cells, neural stem cells, neuroblastoma cells, oligodendrocytes, oligodendroglioma cells, oral keratinocytes, organ culture cells, osteoblasts, ovarian tumour cells, pancreatic beta cells, pericytes, perineurial cells, root sheath cells, Schwann cells, skeletal muscle cells, smooth muscle cells, stellate cells, synoviocytes, thyroid carcinoma cells, villous trophoblast cells, yolk sac carcinoma cells, oocytes, sperm and embryoid bodies.

In an example, the cells are corneal cells. For example, the cells may be corneal stem cells preferably comprising limbal epithelial cells, i.e. a heterogeneous mixture of stem cells and differentiated cells which is obtainable from the limbus at the edge of the cornea. In other words, a multicellular aggregate comprising corneal stem cells may comprise a mixture of corneal stem cells and cells that have not yet fully committed to a corneal epithelial phenotype.

In another example, the cells include stromal progenitor cells such as corneal fibroblasts (keratocytes) in a differentiated or undifferentiated form. Preferably, these corneal fibroblasts are obtained from the peripheral limbus or from limbal rings.

In another example, the cells are bone marrow cells.

In other examples, the cells are chondrocytes.

In yet other examples, the cells are epithelial cells.

In one example, the multicellular aggregate comprises human adipose-derived stromal cells (hASCs), human induced-pluripotent stem cells (iPSC)-derived cortical neurons, human primary kidney proximal tubule epithelial cells (hPTCs), or human corneal stromal fibroblasts (hCSF).

In a further example, the multicellular aggregate comprises human adipose-derived stromal cells (hASCs), human induced-pluripotent stem cells (iPSC)-derived cortical neurons, human primary kidney proximal tubule epithelial cells (hPTCs), human corneal stromal fibroblasts (hCSF), human keratinocytes, human dermal fibroblasts, HEK-293 cells, or human iPSC-derived hemangioblasts.

Preferably the cells are mammalian cells. In another example, the cells are fish cells.

Non-limiting examples of suitable cell types include human cells, or cells from non-human primates, rodents, rabbits, horses, dogs, cats, sheep, cattle, pigs, fish or birds.

Within the context of the invention, the multicellular aggregate described herein is entrapped or encapsulated in a reversibly cross-linked hydrogel.

As used herein, the term “entrapped” refers to the aggregate being physically captured/trapped by the hydrogel, such that it is not released from the hydrogel (unless for example the cross-linking is reversed such that the hydrogel reverts to a solution). The aggregate may be entrapped by virtue of being completely surrounded by the hydrogel, or it may be entrapped by virtue of the majority (but not all) of the aggregate being surrounded by the hydrogel. In this context, the “majority” refers to at least about 50%, at least about 60%, at least about 70%, at least about 80%, at least about 85%, at least about 90%, at least about 95%, at least about 96%, at least about 97%, at least about 98%, or at least about 99% of the aggregate (by volume) being surrounded by the hydrogel. In this context, “completely surrounded” refers to about 100% of the aggregate (by volume) being surrounded by the hydrogel. The term “entrapped” is particularly relevant to aggregates that are not bound/adherent to a surface such as a solid surface of a receptacle (as described elsewhere herein).

The hydrogel may be a coating that covers/surrounds at least the majority of the aggregate, in order to entrap the aggregate in the hydrogel.

The term “encapsulated” refers to enclosing the multicellular aggregate in the hydrogel. In the context of an unbound multicellular aggregate (i.e. an aggregate that is not bound/adherent to a surface such as a solid surface of a receptacle (as described elsewhere herein), a multicellular aggregate is “encapsulated” by a hydrogel when it is completely surrounded by the hydrogel. In the context of a multicellular aggregate that is bound/adherent to a solid surface, the aggregate is considered “encapsulated” when at least the majority of the unbound (“free”) external surface area of the aggregate is surrounded by the hydrogel. In other words, in this context, encapsulation refers to enclosing available surfaces of the multicellular aggregate in the hydrogel. “Majority” refers to at least about 70%, at least about 80%, at least about 85%, at least about 90%, at least about 95%, at least about 96%, at least about 97%, at least about 98%, or at least about 99% of the available external aggregate surface area being covered by the hydrogel.

The phrases “unbound (“free”) external surface area” and “available external aggregate surface area” refer to the outer surface (periphery) of the aggregate that is not in direct contact with the solid surface. This is also referred to herein as the “available surface(s)”.

The hydrogel may be a coating that covers/surrounds at least the majority of the available surface(s) of the aggregate in order to encapsulate the aggregate in the hydrogel. The term “coating” and its equivalents are used herein to describe a layer of hydrogel. The hydrogel coating may be formed separately from the aggregate and then placed over the aggregate (akin to a blanket) in a manner that encapsulates or entraps the aggregate. In this context, a hydrogel coating may comprise a layer of cross-linked alginate that is formed separately (i.e. spatially separate from) from the aggregate. The hydrogel layer may then be placed upon the surface of the aggregate (e.g. a surface-bound monolayer, bilayer or multilayer aggregate), wherein the hydrogel layer coats the aggregate but is not cross-linked in situ. Alternatively, the hydrogel coating may be formed in situ (i.e. in the presence of the aggregate).

In the context of aggregates comprising one or more cell layers, it should be noted that the aggregate may be attached to a solid surface (of e.g. a receptacle as described herein) via adherence of the basal side of the aggregate to the solid surface only. In other words, in aggregates with a plurality of cell layers, it may be that only one of the cell layers (on the basal side of the aggregate) is adherent to the solid surface, and that by virtue of this adherence, the aggregate as a whole is attached to the solid surface. Such aggregates may also be encapsulated or coated by the hydrogel using the methods described herein.

One or more multicellular aggregates may be entrapped or encapsulated within a single hydrogel, where appropriate. For example, a hydrogel may entrap or encapsulate two or more, three or more, four or more, five or more aggregates.

In some examples of the invention, the concentration of cells in the aggregate(s) that is/are entrapped or encapsulated in the hydrogel is from about 10 to 10⁷ cells/ml hydrogel solution (e.g. for alginate gels maintained under cell culture conditions or under ambient conditions).

As used herein a “reversibly cross-linked hydrogel” refers to a hydrogel that is formed by reversible cross-linking (i.e. the cross-linking can be reversed such that the hydrogel reverts back to a solution). Reversal of the cross-linking enables the entrapped or encapsulated multicellular aggregate(s) to be released from the hydrogel (e.g. at their point of use/after transportation or storage is complete). Examples of reversibly cross-linked hydrogels are well known in the art. Accordingly, suitable hydrogels may readily be identified by a person of skill in the art.

The hydrogel referred to herein comprises a hydrogel-forming polymer having a cross-linked or network structure or matrix; and an interstitial liquid. The hydrogel is capable of suppressing or preventing cell differentiation in aggregates encapsulated or entrapped therein. Preferably, the hydrogel is semi-permeable.

The term “hydrogel-forming polymer” refers to a polymer which is capable of forming a cross-linked or network structure or matrix under appropriate conditions, wherein an interstitial liquid and a multicellular aggregate may be retained within such a structure or matrix. The hydrogel will comprise internal pores.

Initiation of the formation of the cross-linked or network structure or matrix may be by any suitable means, depending on the nature of the polymer.

The polymer will in general be a hydrophilic polymer. It will be capable of swelling in an aqueous liquid. In one example of the invention, the hydrogel-forming polymer is collagen. In this example, the collagen hydrogel comprises a matrix of collagen fibrils which form a continuous scaffold around an interstitial liquid and the entrapped or encapsulated multicellular aggregate. Dissolved collagen may be induced to polymerise/aggregate by the addition of dilute alkali to form a gelled network of cross-linked collagen fibrils. The gelled network of fibrils supports the original volume of the dissolved collagen fibres, retaining the interstitial liquid. General methods for the production of such collagen gels are well known in the art (e.g. WO2006/003442, WO2007/060459 and WO2009/004351).

The collagen which is used in the collagen gel may be any fibril-forming collagen. Examples of fibril-forming collagens are Types I, II, III, V, VI, IX and Xl. The gel may comprise all one type of collagen or a mixture of different types of collagen. Preferably, the gel comprises or consists of Type I collagen. In some examples of the invention, the gel is formed exclusively or substantially from collagen fibrils, i.e. collagen fibrils are the only or substantially the only polymers in the gel. In other examples of the invention, the collagen gel may additionally comprise other naturally-occurring polymers, e.g. silk, fibronectin, elastin, chitin and/or cellulose. Generally, the amounts of the non-collagen naturally-occurring polymers will be less than 5%, preferably less than 4%, 3%, 2% or 1% of the gel (wt/wt). Similar amounts of non-natural polymers may also be present in the gel, e.g. peptide amphiphiles, polylactone, polylactide, polyglycone, polycaprolactone and/or phosphate glass.

In some examples of the invention, the hydrogel-forming polymer is alginic acid or an alginate salt of a metal ion. Preferably, the metal is a Group 1 metal (e.g. lithium, sodium, or potassium alginate) or a Group 2 metal (e.g. calcium, magnesium, barium or strontium alginate). Preferably, the polymer is calcium alginate or sodium alginate or strontium alginate, most preferably calcium alginate.

One factor which determines alginate gel permeability is the mannuronic (M) and guluronic (G) acid contents of the gel. Gels with a high M:G ratio have a small intrinsic pore size. The M:G ratio may be manipulated to increase the permeability of gels as necessary to improve the viability of entrapped or encapsulated multicellular aggregate. In some examples, the G content of the alginate gel is 0-30%. In some examples, the M content is preferably 30-70%. In some preferred examples, the gel is an alginate gel with a M content of 50-70% or 60-70% and the gel additionally comprises or a pore enhancer (also referred to herein as a porogen). In some examples, the pore size increasing agent is hydroxyethyl cellulose (HEC). In this example, HEC may be used in the preparation of the hydrogel; it is then completely, substantially completely or partially removed from the hydrogel prior to use. Preferred concentrations of HEC in the hydrogel (during preparation) include 0.5-3.0% HEC, more preferably 1.0-2.5%, and even more preferably 1.2-2.4% HEC. In some preferred embodiments, the concentration of HEC in the hydrogel (during preparation) is 1.2% or 2.4%. (Concentrations are given as weight %). The HEC may be suspended in the gels as micelles. Removal of the HEC may be attained by washing the hydrogel in a suitable aqueous solvent or buffer, e.g. tissue culture medium.

In some examples of the invention, the hydrogel-forming polymer is an alginate. In some examples, the multicellular aggregates can be coated first with a different hydrogel-forming polymer as described herein followed by a further coating of an alginate. In other examples, the hydrogel-forming polymer is a mixture of alginate and another hydrogel-forming polymer. In some examples, the alginate is modified (e.g. with peptides).

In yet other examples of the invention, the hydrogel-forming polymer is a cross-linked acrylic acid-based (e.g. polyacrylamide) polymer.

In yet further examples, the hydrogel-forming polymer is a cross-linkable cellulose derivative, a hydroxyl ether polymer (e.g. a poloxamer), pectin or a natural gum.

In some examples of the invention, the hydrogel is not thermo-reversible at physiological temperatures, i.e. the sol-gel transition of the hydrogel cannot be obtained at a temperature of 0-40° C.

The structure of the hydrogel may be changed by varying the concentration of the hydrogel-forming polymer in the hydrogel. The structure affects the viability of the aggregate in the hydrogel, the rate of cellular differentiation as well as the robustness of the gel and its handling properties. Preferred concentrations of the hydrogel-forming polymer in the hydrogel are 0.2-5% (weight of polymer to volume of interstitial liquid), and include for example 0.2-0.4%, 0.4-0.5%, 0.5-0.7%, 0.7-1.1%, 1.1-1.3%, 1.3-2.2%, 2.2-2.6%, 2.6-3.0%, 3.0-3.5%, 3.5-4.0%, 4.0-4.5% and 4.5-5.0% (or any combination thereof e.g. 0.2-0.5%, 0.2 to 0.7% etc).

In one example, the viscosity of the non-gelled hydrogel solution is up to 500 mPa·s, Optionally, the viscosity of the non-gelled hydrogel solution is between 5 and 200 mPa·s (preferably between 5 and 100 mPa.$).

In other examples, the concentration of the hydrogel-forming polymer in the hydrogel is above 0.25%, 0.3%, 0.4%, 0.5% or 0.6%. In other examples, the concentration of the hydrogel-forming polymer in the hydrogel is below 5%, 4.5%, 4.0%, 3.5%, 3.0%, 2.6%, 2.4%, 1.5%, 1.4%, 1.3% or 1.2%. In some preferred examples, the concentration of the hydrogel-forming polymer in the hydrogel is about 0.3%, about 0.6% or about 1.2%. In some particularly preferred examples, the concentration of the hydrogel-forming polymer in the hydrogel is about 1%. In some particularly preferred examples of the invention, the hydrogel is formed from about 1% sodium alginate or from about 1% calcium alginate.

In some examples of the invention, the gelling of the hydrogel is facilitated using a compound comprising a multivalent metal cation, e.g. using calcium chloride. In particular, calcium chloride (e.g. 50-200 mM calcium chloride, preferably 75-120 mM calcium chloride) may be used to gel alginate hydrogels.

In other examples, of the invention, an alternative metal chloride is used, e.g. magnesium or barium or strontium chloride. Alternatively, other multivalent cations may be used, e.g. La³⁺ or Fe³⁺.

In some examples of the invention, the gels (preferably alginate gels) additionally comprise CO₂. This may aid cell viability after storage, particularly after storage under chilled conditions. The invention further provides a process for preparing a hydrogel, comprising the step of gelling the hydrogel-forming polymer in the presence of a Group 2 metal salt selected from the group consisting of magnesium and calcium salts.

In some examples of the invention, the hydrogel comprises cross-linked alginate. For example, the hydrogel may comprise cross-linked calcium-alginate, strontium-alginate, barium-alginate, magnesium-alginate or sodium-alginate. In one particular example, the cross-linked alginate is from about 0.5% (w/v) to about 5.0% (w/v) calcium alginate. For example, the cross-linked alginate may be from about 1.0% (w/v) to about 2.5% (w/v), about 1.5% (w/v) to about 2.0% (w/v) calcium alginate, or any range therebetween.

The interstitial liquid may be any liquid in which polymer may be dissolved and in which the polymer may gel. Generally, it will be an aqueous liquid, for example an aqueous buffer or cell culture medium. The liquid may contain an antibiotic. Preferably, the hydrogel is sterile, i.e. aseptic. Preferably, the liquid does not contain animal-derived products, e.g. foetal calf serum or bovine serum albumin.

As used herein, the term “suppressing or preventing cell differentiation” means that the rate of cell differentiation within all or a substantial proportion of the cells within a multicellular aggregate contained within the hydrogel (for a given temperature) is at a lower level than that of control cells in an equivalent multicellular aggregate which are maintained under appropriate tissue culture conditions at the same given temperature and which are not entrapped or encapsulated in a hydrogel. A substantial proportion may be at least 50%, 60%, 70%, 80%, 90% or 95%.

The hydrogels may be produced in any suitable size. For ease of transportation, however, the hydrogels are preferably less than 1000 mm in length, preferably less than 500, 250, 100, or 50 mm in length. The thickness of the hydrogel is generally 0.1-50 mm, preferably 0.1-10 mm, 0.5-5 mm, 1.0-2.0 mm, more preferably about 1.5 mm.

The volume of the hydrogels of the invention is preferably 0.2-100 ml, more preferably 0.2-50 ml, 0.2-25 ml or 0.2-10 ml. In some preferred examples, the volume of the hydrogel of the invention is 0.4-5 ml, preferably 0.4-4 ml, and more preferably 0.4-3 ml. In some examples of the invention, the volume may be about 420 μl or about 2 ml.

In some examples of the invention, the hydrogel is in the form of a thin layer, disc or sheet. Hydrogels in such forms are shown herein to enhance cell viability during hypothermic storage. Preferably, the gel is in the form of a disc or thin layer. The disc may for example, have a diameter of 5-50 mm or 10-50 mm, preferably 10-30 mm, more preferably 15-25 mm, and most preferably about 19 mm. The thickness of the thin layer, disc or sheet is generally 0.1-5 mm, preferably 0.5-2.0 mm, more preferably about 1.0 or 1.5 mm, or about 1, 2, 3, 4 or 5 mm. In some examples, the final volume of hydrogel in the disc is preferably 200 μl to 1 ml, preferably 200-600 μl, preferably 300-500 μl and more preferably 400-450 μl.

With regard to the discs of the invention, the preferred hydrogel polymer concentration is about 1.2% due to the increased structural stability provided by this concentration. Preferably, the hydrogel (e.g. a disc) is an uncompressed hydrogel, i.e. it has not been subjected to an axial compressing force.

Within the context of the invention, the multicellular aggregates that are entrapped or encapsulated by a hydrogel of the invention may be packaged in a sealed receptacle.

As used herein, a “sealed receptacle” refers to a container that can maintain a seal against the continuous flow of gases or liquids. For example, the sealed receptacle may be a water-tight or air-tight container e.g. a plastic container. Non-limiting examples of appropriate sealed receptacles include a sealed vial or cryovial or tissue culture flask, optionally together with appropriate media (e.g. cell culture media). In other examples, the hydrogel may be contained within a sealed bag, optionally with a controlled CO₂ level.

In one example, the sealed receptacle is a cell culture vessel. Optionally, the cell culture vessel is selected from a cell culture tube, a cell culture flask, a cell culture dish or a cell culture plate comprising a plurality of wells. For example, the cell culture plate may be selected from a 4-, 6-, 8-, 12-, 24-, 48-, 96-, 384-, 1536-well cell culture plate. Appropriate cell culture vessels are well known in the art.

The receptacle may be sealed using a lid (e.g. a screw fit lid) or another means (e.g. adhesive film, or tape etc).

### Methods of Preparing a Multicellular Aggregate for Storage or Transportation

The invention also provides a method of preparing a multicellular aggregate comprising a plurality of adjoining cells for storage or transportation from a first location to a second location. The method comprises the steps of:

i) contacting the multicellular aggregate with a hydrogel-forming polymer;
ii) polymerising the polymer to form a reversibly cross-linked aggregate-containing hydrogel wherein the aggregate is entrapped or encapsulated in the hydrogel;
wherein the aggregate-containing hydrogel is packaged in a receptacle for storage or transportation from the first location to the second location and wherein the method comprises sealing the aggregate-containing hydrogel into the receptacle.

Optionally, the aggregate is placed within the receptacle prior to step i) of the method e.g. the hydrogel-forming polymer may be contacted with the multicellular aggregate whilst the multicellular aggregate is located within the receptacle that is suitable for storage or transportation. In this example, the adjoining cells of the multicellular aggregate may be placed into the receptacle (e.g. seeded into the receptacle), optionally wherein the cells may adhere to the receptacle (e.g. form an adherent layer in the receptacle).

Alternatively, the aggregate may be placed within the receptacle after step (i) of the method e.g. the hydrogel-forming polymer may be contacted with the multicellular aggregate (and optionally polymerised as per step ii)) before the multicellular aggregate is placed within the receptacle that is suitable for storage or transportation.

Optionally, the method includes the step of iii) dispatching the sealed receptacle for transportation from the first location to the second location.

A multicellular aggregate may be contacted with a hydrogel-forming polymer using any appropriate means. For example, the multicellular aggregate may be mixed with a solution that contains the hydrogel forming polymer (prior to polymerization/aggregation or prior to cross-linking of a hydrogel-forming polymer).

A multicellular aggregate may be contacted with the hydrogel-forming polymer whilst within a sealable receptacle (such that e.g. once the hydrogel is formed, the receptacle can be sealed ready for storage and/or transportation), or it may be contacted with the hydrogel-forming polymer before the aggregate is placed in a sealable receptacle. Suitable receptacles are described elsewhere herein.

The method then comprises polymerising the aggregate-polymer to form a reversibly cross-linked aggregate-containing hydrogel wherein the aggregate is entrapped or encapsulated in the hydrogel. Methods for polymerising the aggregate-polymer to form a reversibly cross-linked aggregate-containing hydrogel are well known in the art, and differ depending on the polymer used. For example, polymerisation of an alginate solution (to form an alginate hydrogel of the invention) may be induced by a chemical agent such as calcium chloride.

As used herein, the terms “polymerising” and “gelling” the hydrogel are used interchangeably to refer to the change in state of the hydrogel-forming polymer from a liquid to a hydrogel.

The hydrogel is gelled under appropriate cell-compatible conditions, i.e. conditions which are not detrimental or not significantly detrimental to the viability of the cells.

In some examples, the hydrogels are prepared under cGMP (current Good Manufacturing Practice) conditions.

For storage, transportation or delivery of the cells in the hydrogel, the hydrogel must be appropriately packaged. The method of the invention therefore comprises packaging the aggregate-containing hydrogel in a receptacle for storage or transportation from the first location to the second location and sealing the receptacle. Suitable receptacles have been described elsewhere herein.

The aggregate-containing hydrogel may be in contact with (e.g. fully or partially immersed in) an appropriate media in the sealed/sealable receptacle. Suitable media include cell or tissue culture media, e.g. supplemented DMEM media.

The method may optionally comprise dispatching the sealed receptacle for transportation from the first location to the second location. As used herein, “dispatching” refers to releasing the receptacle for transport (e.g. releasing the receptacle to the postman for transport/delivery to the intended destination). Dispatch therefore does not include transport of the sealed receptacle to the second location per se.

### Methods of Transporting/Fulfilling an Order for an Aggregate

The invention further provides a method of transporting a multicellular aggregate comprising a plurality of adjoining cells from a first location to a second location. The method comprises the steps of:

(a) preparing the multicellular aggregate for transportation according to the preparation method described elsewhere herein;
(b) transporting the multicellular aggregate of step (a) from the first location to the second location; and optionally
(c) releasing the multicellular aggregate from the hydrogel at the second location.

Furthermore, a method for fulfilling an order or request for a multicellular aggregate is also provided, the method comprising the steps of:

a) receiving an order or request for a multicellular aggregate;
b) preparing the multicellular aggregate for transportation according to the preparation method described elsewhere herein;
c) dispatching the multicellular aggregate of step (b) for transportation; or transporting the multicellular aggregate of step (b) to the location specified in the order or request.

The order or request may be received by any suitable means, e.g. via the internet, email, text-message, telephone or post.

Aspects of the invention described elsewhere (e.g. suitable receptacles, hydrogels aggregates, polymerisation agents) apply equally here.

The aggregates of the invention may be transported within the hydrogel (and sealed receptacle) by any suitable means, e.g. by post or courier, which might include transportation by automotive means, e.g. by car, van, lorry, motorcycle, aeroplane, etc. Preferably, the transportation is by post or courier.

The second location is preferably a location which is remote from the first location, e.g. at least 1 mile, preferably more than 5 miles, from the first location.

Transportation from a first location to a second location may take at least 1 hour, at least 2 hours, at least 5 hours, at least 12 hours, at least 24 hours etc.

The aggregates may be stored or transported within the hydrogel (and the sealed receptacle) at a temperature ranging from −80° C. to 45° C., preferably at 4 to 45° C. in one example, the multicellular aggregate is transported from the first location to the second location at ambient temperature.

In some examples, the aggregates within the hydrogels (and sealed receptacle) are stored or transported under cell culture conditions (e.g. about 37° C., about 5% CO₂ and about 95% humidity). In some examples, they are stored or transported under chilled conditions, e.g. 4-6° C., preferably about 4° C. In a particular example, they are refrigerated when stored or transported (which is defined as from 2-8° C. (EU Pharmacopoeia)). In another example, they are stored or transported cool (defined as from 8-15° C.)).

In other examples, they are stored or transported under ambient conditions, e.g. 10-25° C., preferably 15-20° C. In some examples, the ambient temperature may be up to 30° C. (i.e. 10 to 30° C.), or even up to 40° C. In yet other examples, they are stored or transported at about 37° C.

In some examples, they are stored or transported at Controlled Room Temperature (CRT) (which is defined as from 15 to 25° C.). They may be stored or transported cool or at CRT (i.e. from 8 to 25° C.).

In yet other examples, they are stored or transported at hypothermic temperatures (i.e. below about 35° C., typically in the range of 0 to 32° C.). In one example, they are stored or transported between CRT and 32° C. (i.e. 15 to 32° C.). In another example, they are stored or transported cool, at CRT or up to 32° C. (i.e. from 8 to 32° C.).

In some examples of the invention, the hydrogel comprising the multicellular aggregate is frozen prior to storage and/or transportation. This may extend the time during which the cells of the multicellular aggregate are viable post-thawing and/or increase the usable transit-time. Hence the hydrogel may be used in this way as a post-cryoprotectant. For example, the temperature of the hydrogel comprising the aggregate may be reduced to below 0° C., below −15° C. or below −80° C. The hydrogel comprising the multicellular aggregate may or may not be allowed to defrost or thaw, i.e. to increase its temperature to above 0° C. during storage and/or transportation, preferably at a slow, controlled or uncontrolled rate of temperature increase. In other examples the hydrogels of the invention are not chilled or frozen.

The hydrogel with the multicellular aggregate retained therein may be stored and/or transported for up to 10 or 20 weeks. Preferably, the aggregates are stored in the hydrogel for up to 1, 2, 3, 4, 5, 6, 7, 8, 9 or 10 weeks before being released from the hydrogels. More preferably, the aggregates are stored in the hydrogel for up to 1, 2, 3, 4, 5, 6, 7, 8, 9 or 10 days before being released from the hydrogels.

The hydrogel referred to herein is one from which a multicellular aggregate comprising a plurality of adjoining cells can be released. In other words, after the preservation or storage or transport of the multicellular aggregate contained therein, the hydrogel is capable of being dissociated thus allowing the release or removal of all or substantially all of the multicellular aggregate which was previously retained therein (or removal of the dissociated hydrogel from the aggregate, which may be, for example, adhered to the surface of an appropriate receptacle such as a cell culture plate.

The hydrogel is dissociated under appropriate cell-compatible conditions, i.e. conditions which are not detrimental or not significantly detrimental to the cells and or the integrity of the cells membrane.

Preferably, the hydrogel is dissociated by being chemically disintegrated or dissolved. For example, alginate gels may be disintegrated in an appropriate alginate dissolving buffer (e.g. 0.055 M sodium citrate, 0.15 M NaCl, pH 6.8).

Preferably, at least 50%, 60% or 70% of the cells in the multicellular aggregate remain viable after storage, more preferably at least 80%, 85%, 90% or 95% of the cells remain viable after storage. Viability may be assessed by Trypan blue exclusion assay or other similar means. Other similar means include the MTT (3-(4,5-dimethylthiazol-2-yl)-2,5-diphenyltetrazolium bromide) assay and examination of cell colony formation post-extraction.

### Methods for Storage of a Multicellular Aggregate

In a further aspect, there is provided a method of storing an in vitro multicellular aggregate comprising a plurality of adjoining cells for at least 24 hours, the method comprising the steps of:

(a) preparing the multicellular aggregate for storage by;
i) contacting the multicellular aggregate with an alginate hydrogel-forming polymer;
ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
   (b) storing the packaged multicellular aggregate of step (a) for at least 24 hours at a temperature from 10 to 30° C.

The hydrogel with the in vitro multicellular aggregate retained therein may be stored for up to 10 or 20 weeks. Preferably, the aggregates are stored in the hydrogel for up to 1, 2, 3, 4, 5, 6, 7, 8, 9 or 10 weeks before being released from the hydrogels. More preferably, the aggregates are stored in the hydrogel for up to 1, 2, 3, 4, 5, 6, 7, 8, 9 or 10 days before being released from the hydrogels.

In some examples, the aggregates within the hydrogels (and sealed receptacle) are stored under cell culture conditions (e.g. about 37° C., about 5% CO₂ and about 95% humidity). In some examples, they are stored under chilled conditions, e.g. 4-6° C., preferably about 4° C. In a particular example, they are refrigerated when stored (which is defined as from 2-8° C. (EU Pharmacopoeia)). In another example, they are stored cool (defined as from 8-15° C.)).

In other examples, they are stored under ambient conditions, e.g. 10-25° C., preferably 15-20° C. In some examples, the ambient temperature may be up to 30° C. (i.e. 10 to 30° C.), or even up to 40° C. In yet other examples, they are stored or transported at about 37° C.

In some examples, they are stored at Controlled Room Temperature (CRT) (which is defined as from 15 to 25° C.). They may be stored cool or at CRT (i.e. from 8 to 25° C.).

In yet other examples, they are stored at hypothermic temperatures (i.e. below about 35° C., typically in the range of 0 to 32° C.). In one example, they are stored between CRT and 32° C. (i.e. 15 to 32° C.). In another example, they are stored cool, at CRT or up to 32° C. (i.e. from 8 to 32° C.).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (194); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1: Materials and Methods

### Preservation of Cell Layers in Culture Vessels

Human adipose-derived stromal cells (hASCs), human induced-pluripotent stem cell (iPSC)-derived cortical neurons, and human primary kidney proximal tubule epithelial cells (hPTCs) were cultured using standard protocols and allowed to establish monolayers prior to preservation. Culture plates were removed from normal culture conditions and allowed to equilibrate to room temperature before removing spent medium and replacing with either 300 μL culture medium (− hydrogel control) or coating cells with a 300 μL 1% (w/v) calcium alginate composite. Briefly 1% (w/v) sodium alginate diluted in culture medium was applied over the cells before crosslinking the gel for 20 minutes using 0.1 M calcium chloride. All preparation was conducted at room temperature. Following a 5-minute wash with culture medium, plates were sealed with adhesive films before storing in either a 15° C. controlled temperature incubator, or in 15-25° C. controlled room temperature (CRT)-packaging. Following storage gels were dissolved using 300 μL 0.1 M Trisodium citrate and replaced with medium before return to standard culture conditions.

### Preservation of Spheroids and Tissues in Tightly-Sealed Tubes

hASC-derived spheroids and human corneal stromal fibroblast (hCSF)-derived tissue constructs were encapsulated in 1.2 and 2.4% (w/v) calcium alginate discs, respectively, before storage in tightly-sealed tubes containing culture medium. − hydrogel controls were suspended in culture medium with no alginate hydrogel. Briefly spheroids and tissue constructs were suspended in sodium alginate before crosslinking the gel for 8 minutes using 0.102 M calcium chloride. Gels were then placed in 2 mL cryogenic vials filled with 1.5 mL culture medium before storing in a refrigerator (4° C.) or temperature-controlled incubator (15° C.). Following storage, gels were dissolved using 0.1 M trisodium citrate and spheroids and tissue constructs were placed in culture medium before return to normal culture conditions.

### Preservation of Spheroids in Culture Vessels

hASC-derived spheroids were suspended in 1% (w/v) sodium alginate before gelation in 96-well culture plates as described in 2.1. Plates were sealed and stored at 15° C. in a controlled-temperature incubator before gel dissolution and return to normal culture conditions. − Hydrogel controls consisted of wells filled with 300 μL culture medium.

### Assessment of Viable Recovery

Viable cell recovery, cell viability, and cell morphology were assessed after storage and return to normal culture conditions. Viable cell number was enumerated using AlamarBlue metabolic activity and % viable cell recovery was presented relative to the non-stored control. Viability and morphology was assessed by calcein-AM/ethidium homodimer-1 (live/dead) staining and imaged by fluorescent microscopy.

### Example 2: In-Plate Preservation of Cell Monolayers

Storage Human Adipose-Derived Mesenchymal Stem Cells (ASCs) Preserved in 96-Well Culture Plates

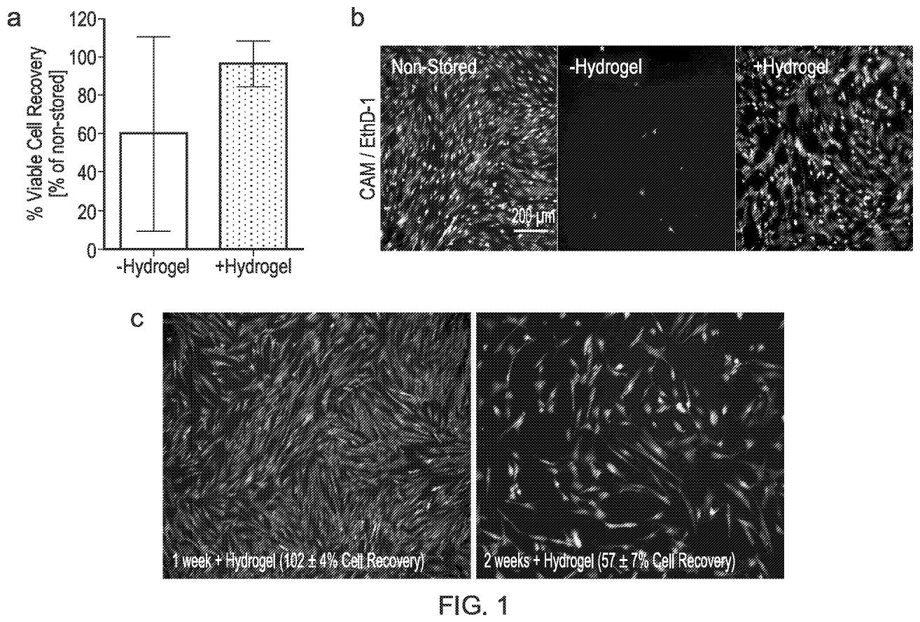

FIG. 1 shows cell recovery, viability and morphology of human adipose-derived mesenchymal stromal cells (hASCs) following storage of cell monolayers in 96-well plates, with or without alginate hydrogel protection. hASCs were seeded in 96-well plates and cultured for 24 hours. Prior to storage, culture medium was removed and replaced with 300 μL culture medium (− Hydrogel) or 300 μL calcium alginate hydrogel composite (+ Hydrogel) before sealing plates and storing at 15° C. (plates illustrated in a). After storage for 3 days, plates were returned to normal culture conditions for 2 hours before assessing % Viable Cell Recovery by AlamarBlue metabolic activity reagent (b) and viability and morphology by calcein-AM (live indicator; green)/EthD-1 (dead indicator; red) staining (c). Where the recovery of hASCs without alginate hydrogel protection was highly variable between experimental set ups, the viability and integrity of ASC monolayers was maintained with alginate hydrogel protection. hASCs were prepared in the same manner with alginate hydrogel protection, and stored for extended periods (1 and 2 weeks) before returning plates to normal culture conditions overnight (d). Even over extended storage periods, a good level of Viable Cell Recovery was observed and cells exhibited a normal spindle-shaped morphology. Results are expressed as means±SD of % cell recovery compared to non-stored cultures.

Storage and Shipping of Human iPSC-Derived Cortical Neurons Preserved in 96-Well Culture Plates

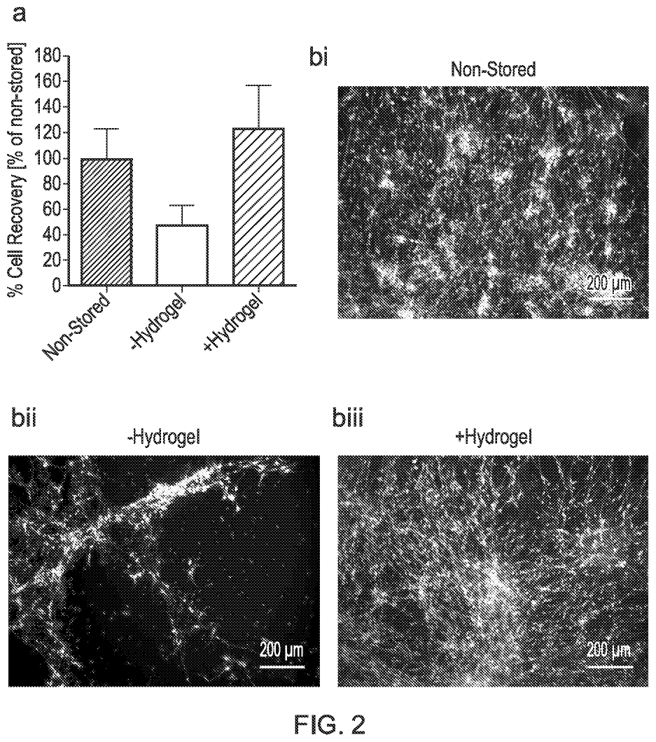

FIG. 2 shows cell recovery, viability and morphology of mature cortical neurons following storage and shipment in 96-well plate, with or without alginate hydrogel protection. Human iPSC-derived differentiated neurons (matured for 31-36 days) were stored and shipped in sealed 96-well plates either with 300 μL neural maintenance medium (− Hydrogel) or coated with 300 μL calcium alginate hydrogel composite (+ Hydrogel). Following overnight storage at 15° C., plates were return-shipped in 15-25° C. controlled room temperature (CRT) packaging (total storage time: 3 days; temperature on arrival: 19° C.). Plates were returned to normal culture conditions for 5 days, before assessing viable cell recovery by AlamarBlue (a). Cells were subsequently stained with calcein-AM (live indicator; green) and ethidium homdimer-1 (dead indicator; red) (b). Storage and shipment without alginate-hydrogel protection resulted in a considerable loss in viable cell number, whilst cell recovery was maintained with when cultures were coated with alginate. Moreover, cultures maintained their morphology and axonal connectivity demonstrating that alginate hydrogels were able to protect cells during room temperature storage and protect against the mechanical stresses induced during transport. Results are expressed as means±SD of % cell recovery compared to non-stored cultures.

### Storage of Human Kidney Proximal Tubule Cell Monolayers Preserved in 96-Well Culture Plates

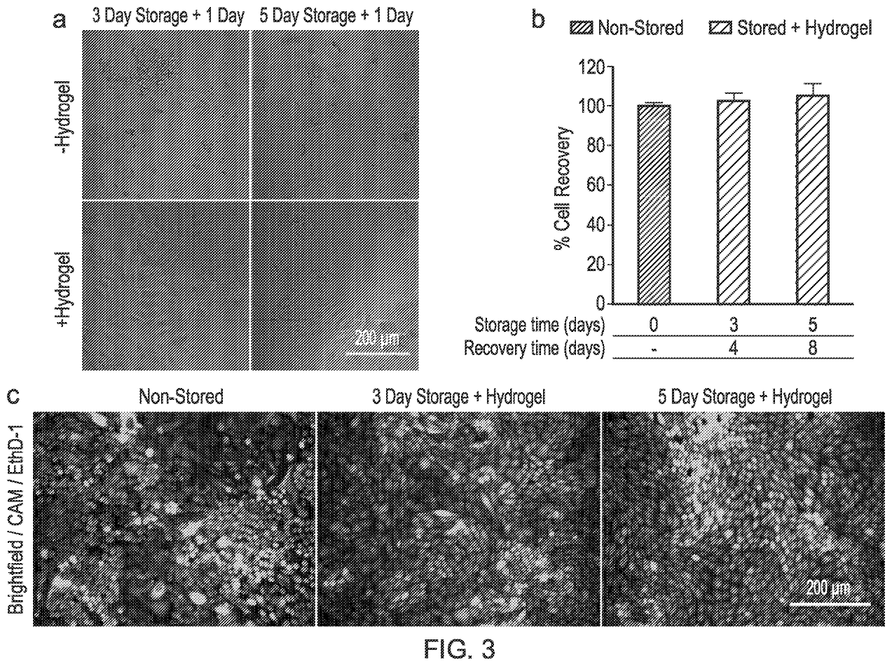

FIG. 3 shows cell recovery, viability and morphology of primary human kidney proximal tubule epithelial cells (hPTCs) following storage in 96-well plates, with or without alginate hydrogel protection. hPTCs from 2 donors were seeded in 96-well plates and cultured for 7 days to reach confluence. Cells were stored for either 3 or 5 days at 15° C. in sealed 96-well plates either with 300 μL culture medium (− Hydrogel) or coated with 300 μL calcium alginate hydrogel composite (+ Hydrogel) before return to normal culture conditions. After 24 hours, without alginate hydrogel protection, there was little evidence of attached viable cells (a). Conversely, culture covered with alginate hydrogels exhibited recovery of a considerable number of viable cells. After a recovery culture period of 3-4 days (for 3-day stored cells) and 7-8 days (for 5-day stored cells), cultures regained full % cell recovery (b) as assessed by AlamarBlue metabolic activity assay. Recovered hPTC cultures formed tight epithelial cultures with high % viability as assessed by calcein-AM (live indicator; green) and ethidium homdimer-1 (dead indicator; red) staining. Results are expressed as means±SD of % cell recovery compared to non-stored cultures.

### Storage of Dermal Keratinocyte Epithelial Cells Preserved in 96-Well Culture Plates

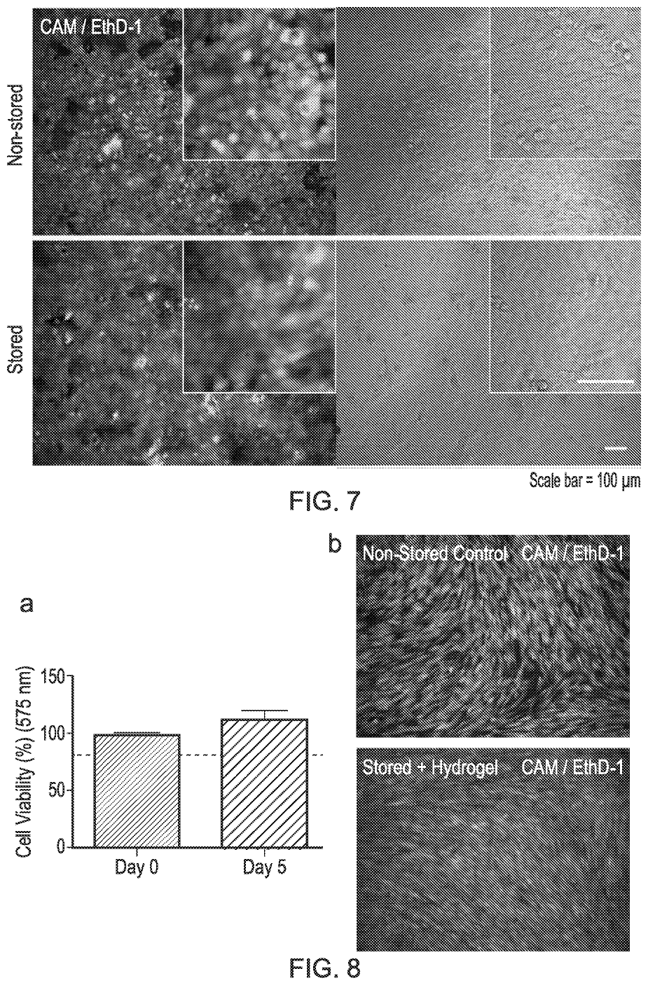

FIG. 7 shows preservation of viability and morphology of human dermal keratinocyte epithelial cells in 96-well culture plates. Keratinocytes from 3 donors were seeded in 96-well plates and cultured until they were sub-confluent. Cells were then overlaid with 300 μL calcium alginate hydrogel composite and stored for 5 days at 15° C. Following gel removal, cells were returned to normal culture conditions overnight and viability and morphology were assessed by live/dead (CAM/EthD-1) staining and fluorescent microscopy. Cells maintained a high cell viability and normal morphology following storage.

### Storage and Shipment of Dermal Fibroblast Cells Preserved in 96-Well Culture Plates

FIG. 8 shows preservation of viability and morphology of human dermal fibroblast cells in 96-well culture plates. Dermal fibroblasts from 3 donors were seeded in 96-well plates and cultured until they were sub-confluent. Cells were then overlaid with 300 μL calcium alginate hydrogel composite and stored for 5 days at 15° C. Following gel removal, cells were returned to normal culture conditions overnight and viability was assessed by MTT assay (a). and live/dead (CAM/EthD-1) staining with fluorescent microscopy. (b). Cells maintained a high cell viability and normal morphology following storage.

### Storage and Shipment of HEK-293 Cells Preserved in 96-Well Culture Plates, 384-Well Culture Plates, and 3D Microscaffolds in 96-Well Plates

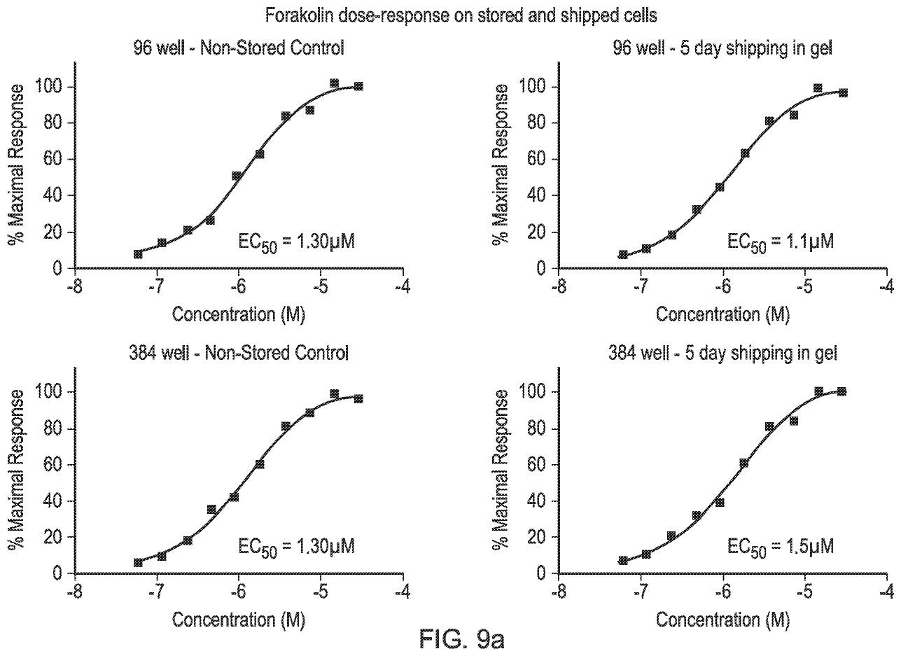

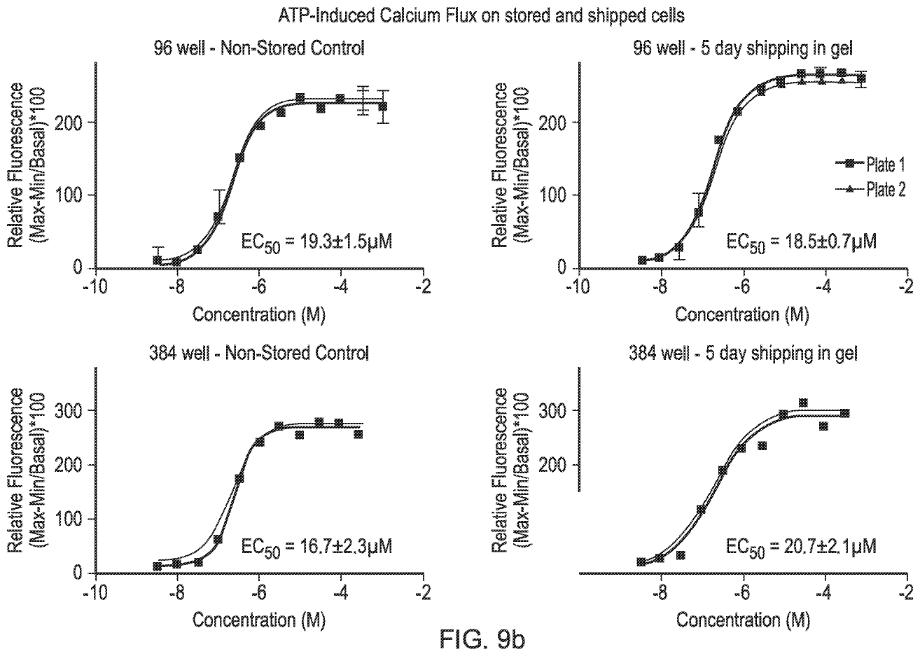

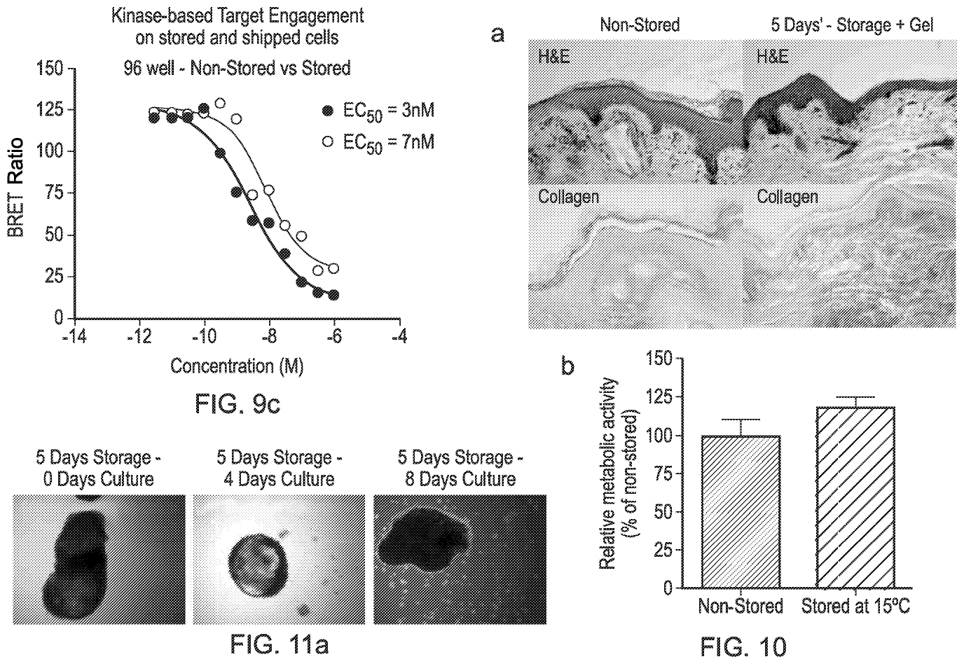

FIG. 9 shows preservation of the pharmacological responsiveness of HEK-293 and transiently transfected HEK-293 cells. HEK-293 cells were seeded for 24 hours in either 96-well plates, 384-well plates before being overlaid with a calcium alginate composite. Cells were then shipped to a remote location (greater than 1 mile) at Controlled Room Temperature and the gel was removed after 5 days of storage. Cells were returned to normal culture conditions overnight before assessing cells for pharmacological responsiveness to Forskolin using a cyclicAMP response element-based luciferase assay (a), and ATP using a calcium fluxbased FLIPR assay (b). EC50 values were similar between non-stored and non-stored cells indicating no loss in function. HEK-293 cells were also transiently transfected with a cDNA encoding the DDR1 kinase sequence prior to encapsulation, storage and shipment over 5 days. Following return to normal culture conditions overnight, cells were treated with Dasatinib and the ligand binding activity was assessed by BRET. Cells retained the transient expression of cDNA and exhibited a comparable EC50 for Dasatinib.

### Example 3: Preservation of Cell-Derived Organoids, Tissues and Spheroids

### Storage of Human ASC Spheroids Suspended in Cryovials

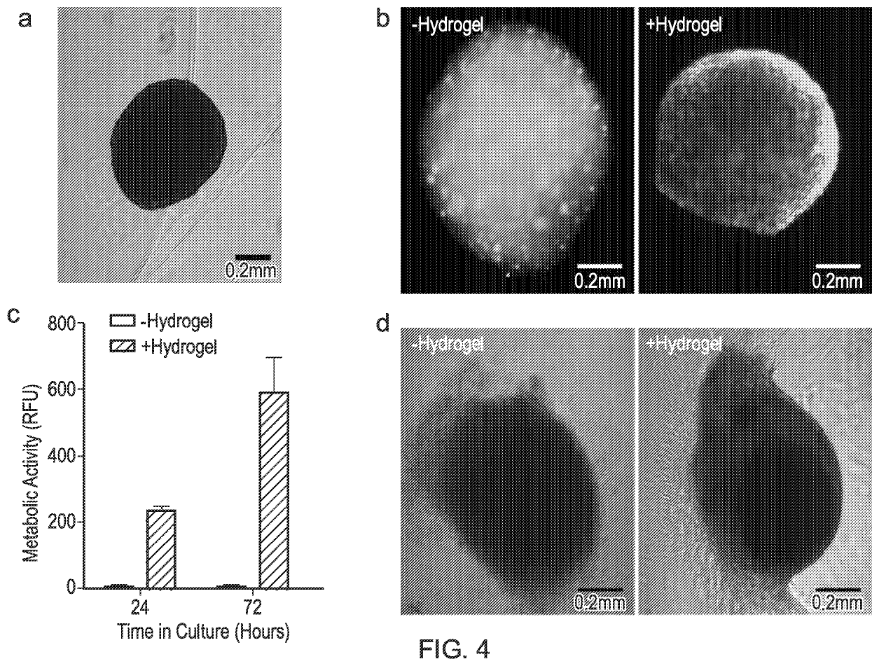

FIG. 4 shows viability of hASC-derived spheroids following storage in tightly-sealed tubes, with or without alginate hydrogel protection. Spheroids consisting of 5×10⁴ hASCs were cultured for 24 hours before suspending in storage medium (− Hydrogel) or encapsulating in 1.2% (w/v) calcium alginate (+ Hydrogel). Spheroids were placed in tightly-sealed vials containing storage medium and stored for 72 hours at 4° C. Spheroids were assessed for viability after release from storage before returning to normal culture conditions. a: Image of a hASC spheroid embedded in alginate; b: Calcein-AM/Ethidium Homodimer-1 (live/dead) staining of spheroids following storage; c: Relative metabolic activity of spheroids following return to normal culture conditions for 24 or 72 hours; d: Images of stored spheroids after 72 hours in culture. Without encapsulation, spheroids appeared swollen and were unable to attach and recover metabolic activity upon return to normal culture conditions. Alginate-encapsulation prevented this and preserved the viability and integrity of hASC-derived spheroids. Results are expressed as means±SD.

### Storage of ASC Spheroids Preserved in 96-Well Culture Plates

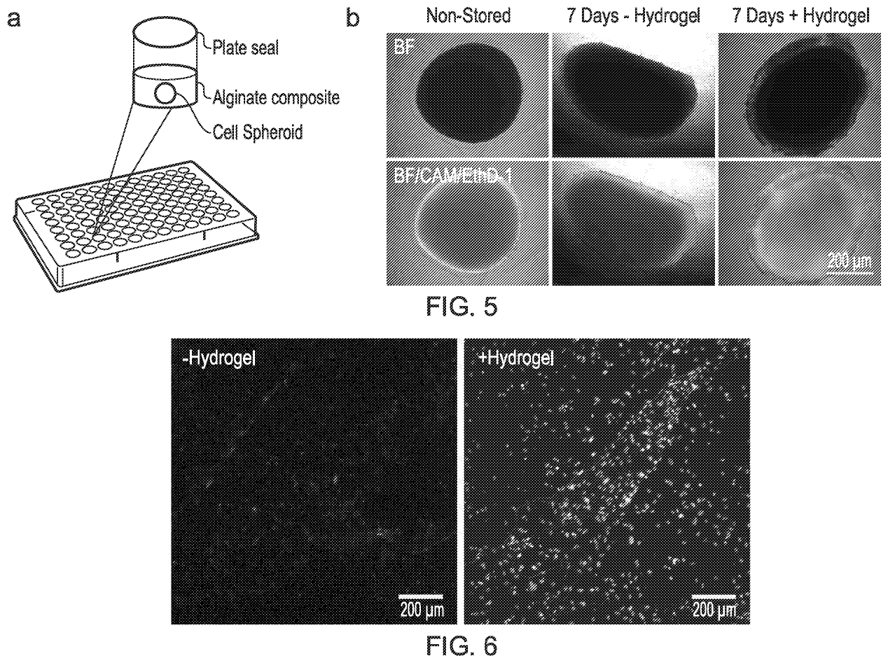

FIG. 5 shows viability of hASC-derived spheroids following storage in 96-well plates, with or without alginate hydrogel protection. Spheroids consisting of 7×10⁴ hASCs were cultured for 24 hours before suspending in storage medium (− Hydrogel) or encapsulating in calcium alginate (+ Hydrogel) in sealed 96-well plates (as illustrated in a). Culture plates were stored for 7 days at 15° C. before return to normal culture conditions, without alginate hydrogel removal. After 24 hours in culture, those spheroids that were not encapsulated demonstrated very poor viability as assessed calcein-AM (live indicator; green) and ethidium homdimer-1 (dead indicator; red) staining (b). On the contrary spheroids with alginate protection remained viable.

### Storage of Human Corneal Stromal Fibroblast-Derived Tissue Constructs

FIG. 6 shows viability and integrity of human corneal stromal fibroblast (hCSF) constructs in tightly-sealed tubes, with or without alginate hydrogel protection. hCSF-derived tissue constructs were either suspended in storage medium (− Hydrogel) or encapsulated in calcium alginate (+ Hydrogel). Tissues were placed in tightly-sealed tubes containing storage medium and stored for 72 hours at 15° C. Tissues were assessed for viability after release from storage by Calcein-AM/Ethidium Homodimer-1 (live/dead) staining. Without encapsulation, no live cells could be identified and total cell number was low, but encapsulation during storage maintained cell viability and tissue integrity.

### Storage of Human Abdominal Skin Biopsies in 96-Well Plates

FIG. 10 shows preservation of freshly collected abdominal skin biopsies in 96-well plates. Fresh skin biopsies were isolated, dissected, and placed in 96-well plates before being overlaid with a calcium alginate composite. Skin was stored for a period of 5 days at 15° C. before removing the gel and returning to culture for 4 hours. Subsequently, tissue integrity was examined by H&E and collagen staining (a) and viability was examined by looking at relative metabolic activity by alamarBlue (b). Tissues stored for 5 days exhibited no change in the structure or integrity, and no loss in viability.

Storage of iPSC-Derived Hemangioblasts (Macrophage Progenitor Factories)

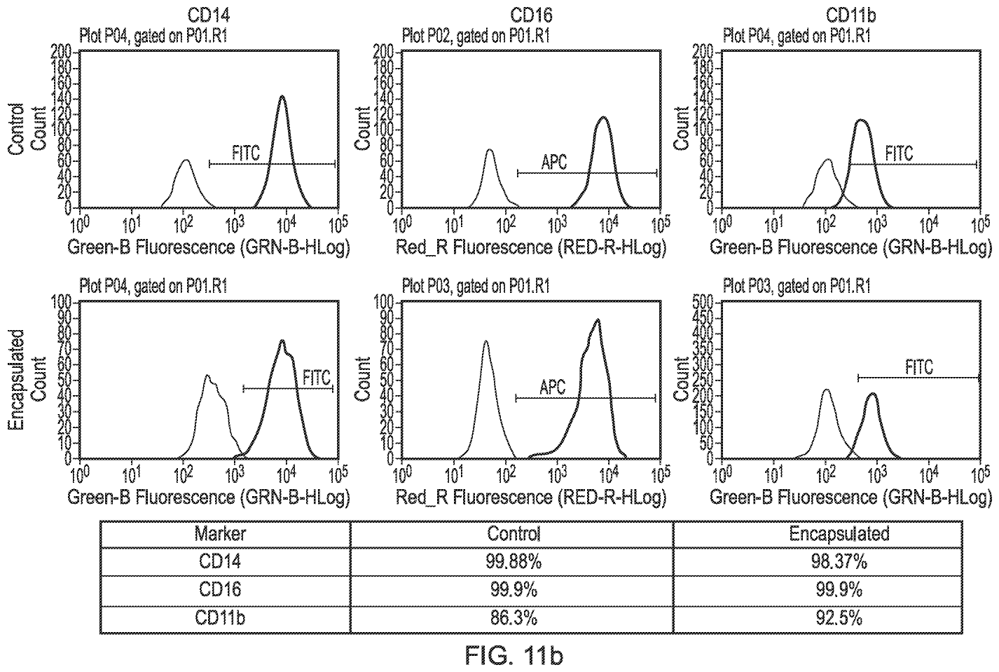

FIG. 11 shows preservation of iPSC-derived hemangioblasts suspended in calcium alginate hydrogel beads. Hemangioblasts were suspended in sodium alginate before crosslinking with calcium in the form of beads. Beads suspended in complete medium were shipped to a remote site at controlled room temperature over a period of 5 days. Hemangioblasts were retrieved from alginate beads and returned to culture for a period of 20 days, over which time macrophage progenitor cells were collected and assessed for phenotype. Encapsulation preserved the capacity for hemangioblasts to produce macrophage progenitors which expressed typical lineage markers.

### Storage of Human Skin 3D Constructs

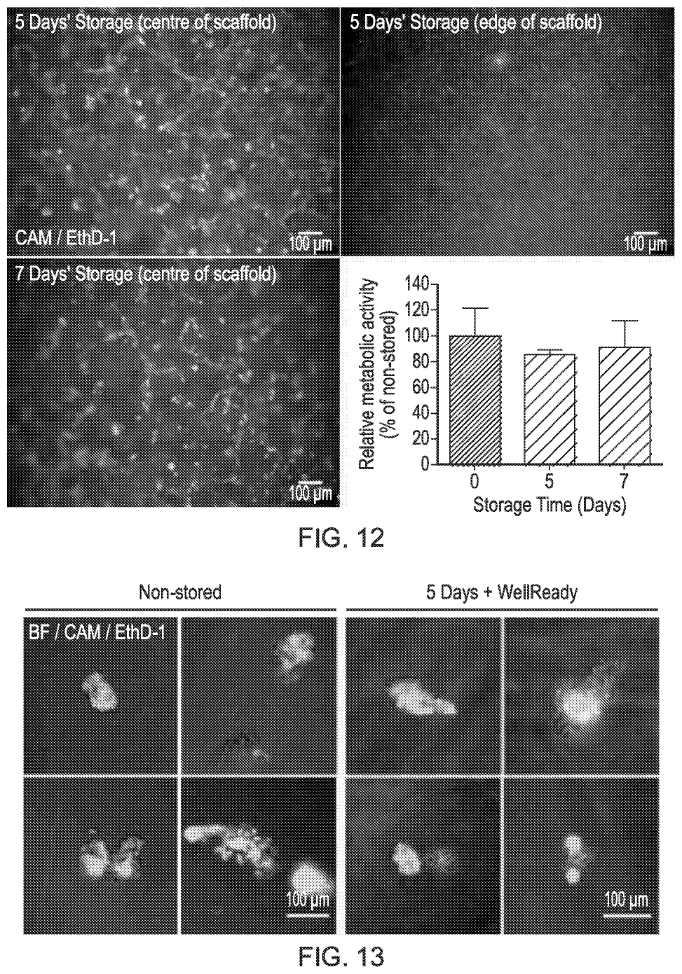

FIG. 12 shows preservation of human skin 3D constructs with alginate hydrogel protection. 3D tissue constructs comprised of dermal keratinocytes and fibroblasts in 3D culture inserts were stored and shipped with alginate hydrogel protection over a 5- and 7-day period at Controlled Room Temperature. After gel removal and overnight incubation, cell viability was assessed. Live cells (CAM-positive; green) were seen throughout the scaffold with little evidence of dead cells following 5 and 7 days' storage and shipment Relative metabolic activity of skin models was maintained after storage for both 5 and 7 days (approximately 90% of the non-stored control).

### Storage of Colorectal Cancer Organoids Preserved in 96-Well Culture Plates

FIG. 13 shows preserved viability and morphology of colorectal cancer organoids following storage in 96-well plates with alginate hydrogel protection. Colorectal cancer organoids were established in culture in 96-well plates. Organoids were then were then overlaid with 150 μL calcium alginate hydrogel composite and stored for 5 days at 15° C. Following gel removal, cells were returned to normal culture conditions overnight and viability and morphology was assessed by live/dead (CAM/EthD-1) staining with fluorescent and brightfield microscopy. Organoids maintained a high cell viability and normal morphology following storage.

### Example 4: Technical Summary

The data presented here describes the use of alginate as a layer or coating for the preservation of cells and simple tissues during storage and/or transport. It presents the preservation of cell layers in situ (i.e. in the culture vessel in which they are seeded and/or grown). Cells preserved in this manner include stromal cells, epithelial cells and neuronal cells. Also presented are data describing the preservation of simple multicellular spheroids and simple 3D tissue constructs. Data demonstrates the capacity for alginate hydrogel coating to preserve cell viability and culture/tissue integrity during room temperature storage, as well as offer mechanical protection during transport.

1. A method of transporting an in vitro multicellular aggregate comprising a plurality of adjoining cells from a first location to a second location, the method comprising the steps of:
(a) preparing the multicellular aggregate for transportation by;
i) contacting the multicellular aggregate with an alginate hydrogel-forming polymer; ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
(b) transporting the packaged multicellular aggregate of step (a) from the first location to the second location at a temperature from 10 to 30° C., wherein the distance between the first and second location is at least 1 mile.

2. The method of claim 1, further comprising:
(c) releasing the multicellular aggregate from the alginate hydrogel at the second location.

3. A method for fulfilling an order or request for an in vitro multicellular aggregate comprising a plurality of adjoining cells, the method comprising: receiving an order or request for a multicellular aggregate; and
a) preparing the multicellular aggregate for transportation by;
i) contacting the multicellular aggregate with an alginate hydrogel-forming polymer; ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
b) dispatching the packaged multicellular aggregate of step (a) for transportation; or transporting the multicellular aggregate of step (a) to the location specified in the order or request.

4. The method of claim 3, wherein the multicellular aggregate is transported from the first location to the second location at a temperature from 10 to 30° C. and the distance between the first and second location is at least 1 mile.

5. A method of storing an in vitro multicellular aggregate comprising a plurality of adjoining cells for at least 24 hours, the method comprising the steps of:
(a) preparing the multicellular aggregate for storage by;
i) contacting the multicellular aggregate with an alginate hydrogel-forming polymer; ii) polymerising the polymer to form a reversibly cross-linked aggregate-containing alginate hydrogel wherein the multicellular aggregate is entrapped or encapsulated in the alginate hydrogel; and
iii) packaging and sealing the multicellular aggregate-containing alginate hydrogel in a water tight or air tight receptacle; and
(b) storing the packaged multicellular aggregate of step (a) for at least 24 hours at a temperature from 10 to 30° C.

6. The method of claim 5; further comprising:
(c) releasing the multicellular aggregate from the alginate hydrogel after storage.

7. The method of claim 5, wherein step (a) comprises placing the multicellular aggregate in the receptacle for transportation, dispatch or storage prior to contacting the multicellular aggregate with the alginate hydrogel-forming polymer.

8. The method of claim 5, wherein step (a) comprises placing the multicellular aggregate in the receptacle for transportation, dispatch or storage after contacting the multicellular aggregate with the alginate hydrogel-forming polymer.

9. The method of claim 5, wherein the receptacle is a sealed storage vial or transport tube, or wherein the receptacle is a cell culture vessel.

10. The method as of claim 9, wherein the receptacle is a cell culture vessel selected from a cell culture tube, a cell culture flask, a cell culture dish, or a cell culture plate comprising a plurality of wells.

11. The method as of claim 10, wherein the receptacle is a cell culture plate comprising a plurality of wells selected from a 4-, 6-, 8-, 12-, 24-, 48-, 96-, 384-, or 1536-well cell culture plate.

12. The method of claim 5, wherein the hydrogel-forming polymer comprises calcium-alginate, strontium alginate, barium-alginate, magnesium-alginate, or sodium-alginate.

13. The method as of claim 12, wherein the alginate is in an amount from 0.5% (w/v) to 5.0% (w/v) calcium alginate.

14. The method of claim 5, wherein the multicellular aggregate comprises a tissue, a cell layer, a spheroid, an organoid, or any combination thereof.

15. The method of claim 5, wherein the multicellular aggregate comprises heterogenous cell types.

16. The method of claim 5, wherein the multicellular aggregate comprises homogenous cell types.

17. The method of claim 5, wherein the multicellular aggregate comprises human cells.

18. The method of claim 5, wherein the multicellular aggregate comprises human adipose-derived stromal cells (hASCs), human induced-pluripotent stem cells (iPSC)-derived cortical neurons, human primary kidney proximal tubule epithelial cells (hPTCs), or human corneal stromal fibroblasts (hCSF).

19. The method of claim 5, wherein polymerisation is induced by a chemical agent.

20. The method of claim 19, wherein the chemical polymerisation agent is calcium chloride.
